(19) United States
(12) Patent Application Publication
(10) Pub. No.: US 2012/0192871 A1
(43) Pub. Date: Aug. 2, 2012

(54) FEMALE CONDOM

(76) Inventors: Alla V.K. REDDY, Plainsboro, NJ (US); Madhusudhan Reddy ALLA, Plainsboro, NJ (US); Raghunatha Reddy ALLA, Plainsboro, NJ (US); Ravikumar Reddy ALLA, Bellevue, WA (US)

(21) Appl. No.: 13/443,993

(22) Filed: Apr. 11, 2012

Related U.S. Application Data
(63) Continuation of application No. 12/542,848, filed on Aug. 18, 2009, now abandoned.
Application No. 12/542,848 is a continuation of application No. 10/664,861, filed on Sep. 22, 2003, now abandoned.
(60) Provisional application No. 60/411,989, filed on Sep. 19, 2002.
(60) Provisional application No. 60/411,994, filed on Sep. 19, 2002.

(51) Int. Cl. A61F 6/06

(52) U.S. Cl. 128/830

(57) ABSTRACT

A female condom having a highly elastic resilient pouch portion is able to stretch from an original contracted configuration into an elongated configuration and then return to the original contracted configuration. The pouch may be fashioned with regular convolutions that act in a spring-like manner for biasing the condom into the contracted configuration. Elastic biasing members may be incorporated into the construction of the condom for providing or augmenting the spring-like action of the condom. The condom of the invention may include a contoured V-shaped outer frame which also may have a spring bias incorporated. The outer frame may flex so as to better retain contact with the anatomy of the female when the condom is inserted in the vagina. A retention sponge is located in the closed end of the pouch and can serve as a resilient elastic member in addition to or in place of the pouch.

### CROSS-REFERENCES TO RELATED APPLICATIONS

This application is a continuation of U.S. patent application Ser. No. 12/542,848, filed Aug. 18, 2009, which is a continuation of U.S. patent application Ser. No. 10/664,861, filed Sep. 22, 2003, now abandoned, which is incorporated by reference in its entirety herein and which claims the benefit of U.S. Provisional Patent Application No. 60/411,989, filed Sep. 19, 2002, and U.S. Provisional Patent Application No. 60/411,994, filed Sep. 19, 2002.

### BACKGROUND OF THE INVENTION

1. Field of the Invention

The invention relates generally to prophylactic devices and, more particularly, to a female condom for insertion into the vagina of a female for protection from pregnancy and disease during sexual intercourse.

2. Description of the Related Art

With the proliferation of sexually transmitted diseases (STDs), including human immunodeficiency virus (HIV), the virus that causes acquired immune-deficiency syndrome (AIDS), and with the uncontrolled growth of the world's population, there is a pressing unmet need for an effective means for preventing the transmission of STDs and for controlling population growth. One method for accomplishing both these goals is by introducing an effective barrier between male and female sex organs during sexual intercourse for preventing the transmission of bodily fluids and for limiting the actual skin-to-skin contact between the sexual organs. Heretofore, conventional means for creating such barriers have included the use of condoms, diaphragms, prophylactic gels, creams, and the like.

Since ancient times, one successful means for creating a barrier to bodily fluids during sex has been the use of male condoms. Male condoms for placement over the penis were originally constructed from animal tissue, and condoms of this type are still in use today. Condoms of animal tissue are generally effective for preventing the passage of semen, but do not prevent the passage of certain microbes, and in particular viruses, such as HIV. Accordingly, most male condoms are now constructed from latex, polyurethane, or other resilient modern polymers that are able to act as a barrier to microbes as well as bodily fluids. While male condoms have become popular for the control of STDs and pregnancy worldwide, there is still much room for improvement. For example, many men do not like to use male condoms because such condoms can deaden sensation, cause erectile dysfunction, inability to ejaculate, or the like. In addition, some men simply refuse to wear condoms.

Because of the shortcomings with male condoms, there have been proposals to provide female prophylactic devices that can be worn by a female prior to sex and disposed of following use, i.e., a female version of a condom. However, at the present, there are few satisfactory female condoms on the market. One reason that the female condoms currently available have not become popular is that they fail to provide adequate protection during sexual intercourse in different positions. For example, the length of the vaginal canal changes depending upon the position of the female partner during intercourse. In particular, in the prone position (e.g., woman on top) the vaginal canal length is shorter because the suspended cervix moves anterior, whereas in the supine position (e.g., woman on bottom) the vaginal canal length is longer because the cervix moves to the posterior position. Since the existing female condoms are not designed to change their length along with the change in length of the vaginal canal during intercourse, they can cause several failures and inconveniences during intercourse.

FIG. 1 illustrates a cross sectional view of a female pelvic region 10 showing the relative locations of the pubic bone 12, urethra 14, bladder 16, vagina 18, uterus 20, cervix 22, and rectum 24. A prior art condom 26 is inserted in vagina 18, and includes an outer ring or frame 28, a tubular protective pouch portion 30 attached to frame 28, and a resilient retaining ring 32 located on the opposite end of pouch 30 from frame 28. Frame 28 remains outside of vagina 18 following insertion of the pouch portion 30, while retaining ring 32 serves to anchor condom 26 in the vaginal canal 18. Retaining ring 32 is an integral part of condom 26 and requires special manufacturing tooling. Furthermore, retaining ring 32 requires careful placement within vaginal canal 18 to ensure that it will fit in an interference relationship therewith to prevent pouch 30 from being withdrawn during coitus. Furthermore, retaining ring 32 can be oriented in a direction that will produce undesirable contact with an erect penis, thereby causing discomfort or pain during intercourse. Furthermore, in some instances, it is possible for frame 28 to enter into vagina 18 during intercourse causing undesirable exposure of the users to bodily fluids or skin-to-skin contact.

In addition, when the length of the vaginal canal shortens, such as during a change in sexual position, as illustrated in FIG. 1, the pouch portion of condom 26 can extend out of vagina 18. This can obstruct the penis (not shown) from entering or reentering vagina 18 during sexual intercourse. The looseness of outer frame 28 because of the long pouch portion 30 of condom 26 can allow the penis to have a false passage into vagina 18 (i.e., the penis can enter vagina 18 on the exterior of condom 26, rather than through outer frame 28 and pouch 30, thus bypassing the condom).

Furthermore, other female condoms known in the art (not shown) have an internally located resilient insert element for holding the pouch portion of the female condom in place, or a series of flexible protrusions on the outside of the pouch that are inclined in a direction to prevent pull-out of the pouch. However, these prior art condoms require use of an insertion tool for inserting the anchor elements. The use of an insertion tool is undesirable since it adds to the inconvenience associated with using the condom.

U.S. Pat. Nos. 5,992,415 and 6,035,853, which include several of the same inventors as in the present application, the disclosures of which are incorporated by reference herein in their entirety, show female condoms that use retention sponges. In these patents, a retention sponge is located in a cavity at the end of a pouch portion for retaining the condom in the vagina. However, in some cases, the retention sponge can invert during intercourse, causing the pouch to pullout. In addition, U.S. Pat. No. 6,035,853 has a telescoped body portion that can prematurely unfold causing the pouch to pullout.

Thus, while suitable for their intended purposes, the prior art female condoms have been found to lack an ease of application and quality of operation that is necessary to make female condoms acceptable and convenient to most users. Accordingly, there is a need for an effective female condom that overcomes the shortcomings associated with the prior art female condoms, male condoms, and other conventional prophylactic barrier-type devices.

### BRIEF SUMMARY OF THE INVENTION

In a first aspect, the invention is directed to a female condom having a highly elastic resilient pouch portion that is able to freely stretch or extend from an original contracted or compacted configuration into an elongated or stretched configuration, and then return under resilient urging to the original contracted configuration. The pouch may be fashioned with regular circular convolutions that act in a spring-like manner for biasing the condom in a longitudinal direction so that the condom will automatically move back into the contracted configuration. In addition, elastic biasing members or strips may be incorporated into the construction of the condom for providing or augmenting the longitudinal spring-like action of the condom. Thus, the pouch of the condom in one embodiment may be a bunched-up pouch having one or more elastic biasing members attached for retaining the condom in a contracted condition. Alternatively, the pouch may be a smooth pouch made of highly resilient natural latex having high elongation characteristics so that the pouch has sufficient spring bias along the length of the pouch to return the condom to its original configuration and position.

In an additional aspect, the condom of the invention may include a contoured V-shaped or triangular outer frame which also has a spring bias incorporated. Thus, the outer frame may include a curved or dome-like shape which enables the frame to flex so as to better stay in contact with the vulva or external anatomy of the female when the pouch of the condom is located in the vagina. The frame is contoured in a concave fashion on the side that contacts the area of the female anatomy surround the vagina. The V-shaped outer frame is sufficiently resilient to flatten out during penetration by the male to provide more intimate contact during intercourse, and then return to its original curved shape following withdrawal.

In yet another aspect, the condom of the invention includes a retention means located at the closed end of the pouch. The retention means of the invention is preferably a sponge which serves to anchor the closed end of the pouch in the vagina. The sponge may be circular or any other desired shape, and can have a diameter larger than, equal to, or smaller than the pouch portion. Advantageously, the sponge is retained within the pouch in a cavity formed at the end of the pouch by a restricted neck which retains the sponge in the cavity. The sponge can also serve as a resilient spring-like member in addition to or in place of the pouch. Thus, the sponge may be made thick in the longitudinal direction, such as one-half to two inches in thickness, so as to collapse when contacted by the penis, and then rebound upon withdrawal. In addition, the sponge may include an insertion opening for receiving a finger of a user to facilitate insertion of the condom into the vagina. Accordingly, a separate applicator is not required for insertion of the condom of the invention into a vagina. These and other features and advantages of the present invention will become apparent to those of ordinary skill in the art in view of the following detailed description of the preferred embodiments.

### BRIEF DESCRIPTION OF THE DRAWINGS

The accompanying drawings, in conjunction with the general description given above, and the detailed description of the preferred embodiments given below, serve to illustrate and explain the principles of the preferred embodiments of the best mode of the invention presently contemplated, wherein:

FIG. 1 illustrates a cross-sectional view of a female's pelvic region showing a prior art condom inserted;

FIG. 2 illustrates a cross-sectional view of a female's pelvic region showing the condom of the invention inserted;

FIG. 3a illustrates a front view of a first embodiment of a condom of the invention;

FIG. 3b illustrates a sectional view of the condom of FIG. 3a taken along line 3b-3b;

FIG. 3c illustrates the condom of FIG. 3b in the elongated position with the frame flattened out;

FIG. 4 illustrates an elastic member of the invention;

FIG. 5a illustrates an alternative placement of elastic member 124 on the condom of FIG. 3a;

FIG. 5b illustrates a sectional view of the condom of FIG. 5a taken along line 5b-5b;

FIG. 6 illustrates an alternative placement of elastic member 124 on the condom of FIG. 3b;

FIG. 7 illustrates an alternative embodiment of the condom of the invention having a bunched pouch;

FIG. 8 illustrates an alternative embodiment of the condom of the invention having a larger sponge and/or a highly resilient non-convoluted pouch;

FIG. 9 illustrates a first preferred sponge configuration of the invention;

FIG. 10 illustrates a second preferred sponge configuration of the invention;

FIG. 11 illustrates a third preferred sponge configuration of the invention; and

FIG. 12 illustrates an alternative embodiment of the condom of the invention having a first pouch containing a second pouch.

### DETAILED DESCRIPTION OF THE INVENTION

In the following detailed description of the invention, reference is made to the accompanying drawings which form a part of the disclosure, and, in which are shown by way of illustration, and not of limitation, specific embodiments by which the invention may be practiced. In the drawings, like numerals describe substantially similar components throughout the several views. The embodiments illustrated are described in sufficient detail to enable those skilled in the art to practice the invention. Other embodiments may be utilized and derived therefrom, such that structural and logical substitutions and changes may be made without departing from the scope of the invention. The following detailed description, therefore, is not to be taken in a limiting sense, and the scope of the invention is defined only by the appended claims, along with the full range of equivalents to which such claims are entitled.

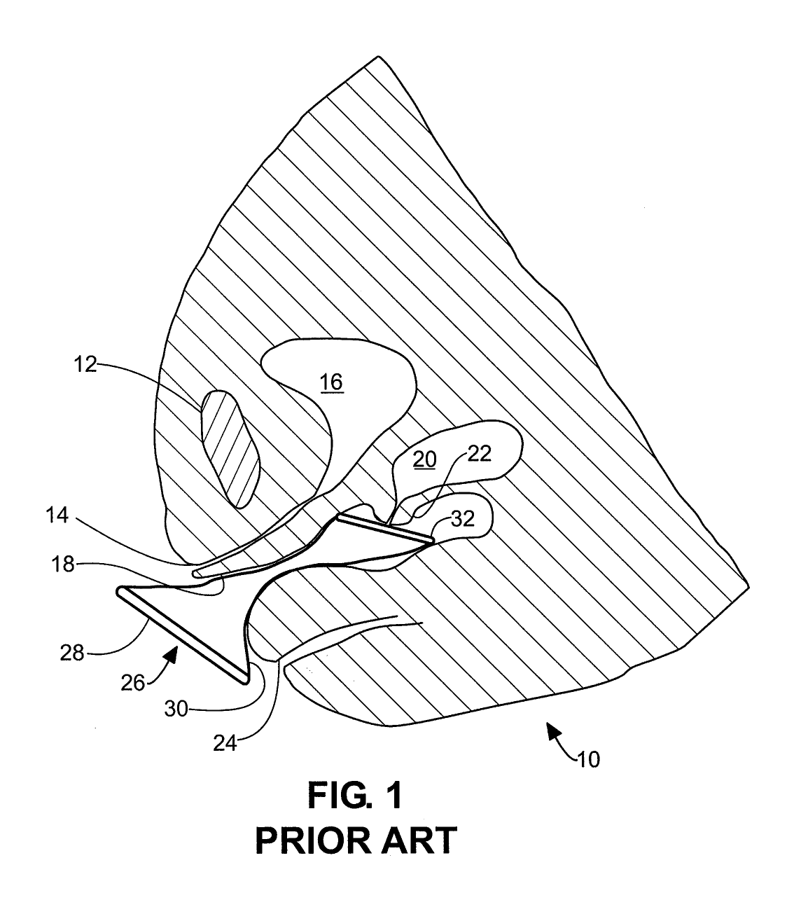

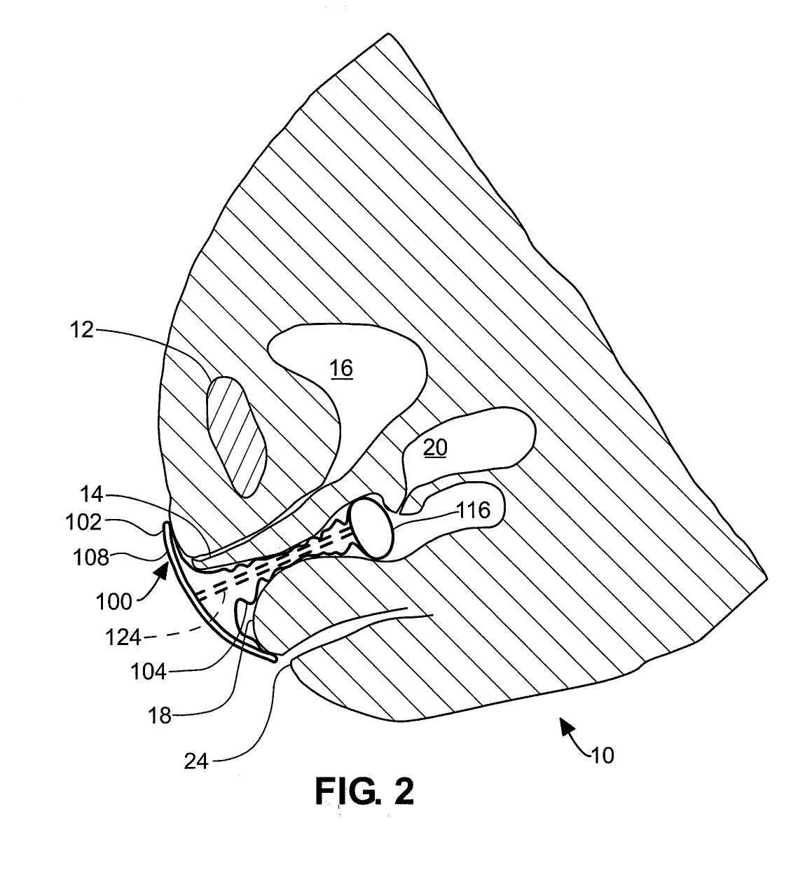

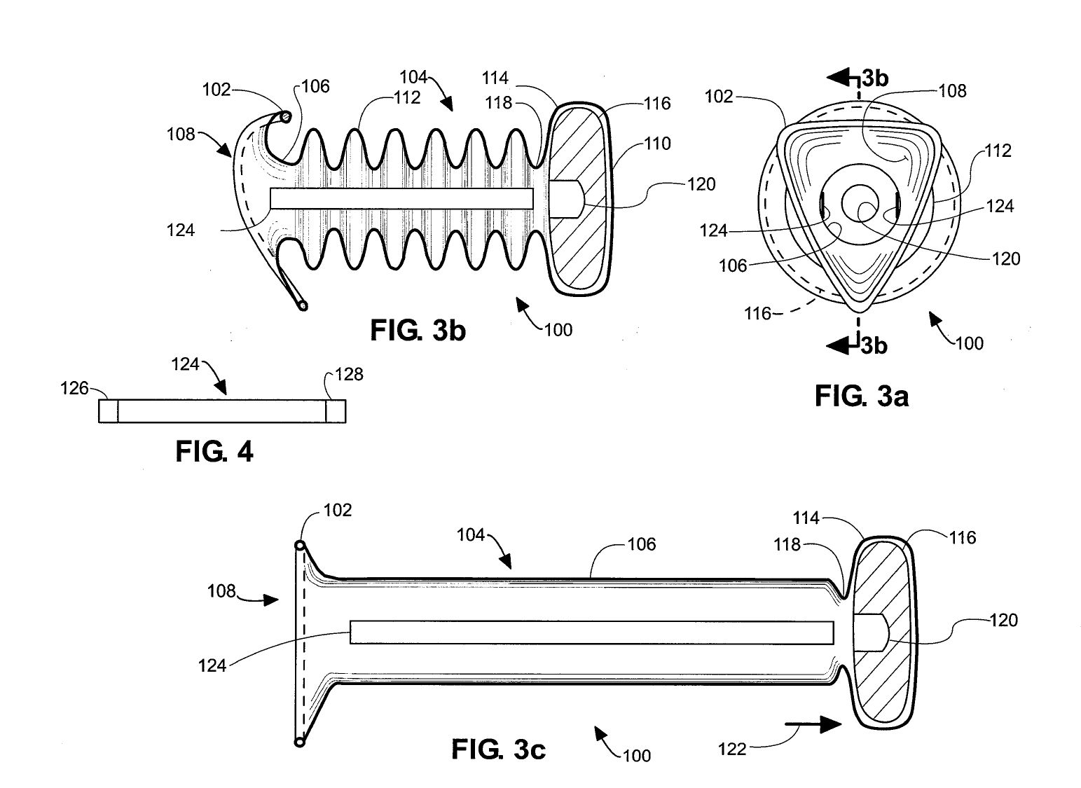

Referring initially to FIG. 2, there is shown a first embodiment of a condom 100 of the invention inserted into a vagina 18 of the female pelvic region 10 described above with respect to FIG. 1. As also illustrated in FIGS. 3a-3c, condom 100 includes a contoured outer frame 102. Frame 102 is advantageously formed in an inverted triangle, or V-shape, as illustrated in FIG. 3a, yet frame 102 is also contoured in curved or dome-like manner, as illustrated in FIG. 3b, so as to better conform to the female anatomy than the prior art condoms. This contour of frame 102 is generally concave on the side of frame 102 that contacts the female anatomy. Frame 102 is formed of any suitable resilient polymeric material that has sufficient flexibility to be easily deformed, yet be stiff and resilient enough to have a spring bias for returning frame 102 its original configuration following removal of any deforming pressure. Frame 102 may be formed by molding or other means well known in the art.

A pouch 104 is attached to frame 102 by rolling and bonding, molding, heat-sealing, or other known methods. Pouch 104 includes a resilient tubular body portion 106 having an open end 108 and a closed end 110. A plurality of bellows-like regular convolutions or corrugations 112 are formed along the length of pouch 104. These convolutions are preferably circular, and are formed to act in a spring-like manner whereby if pouch 104 is extended in the longitudinal direction (i.e., along the major length of the pouch) by the insertion of a penis, or the like, pouch 104 will return to its original non-extended configuration following withdrawal of the penis due to a spring-like bias in the longitudinal direction that is substantially greater than in any prior art female condoms.

Pouch 104 also includes a sponge cavity 114 for retaining a sponge 116 at closed end 110. Sponge cavity 114 includes a neck 118 that is of a smaller diameter than sponge 116, so that sponge 116 is retained within cavity 114. Pouch 104 is preferably formed of natural latex, but may also be formed of other materials having similar suitable properties as will be described in more detail below. Sponge 116 is formed of any suitable soft resilient mass or any sponge-like material and may be of any desired suitable shape, but is preferably circular. The diameter of sponge 116 can be the same as the diameter of the pouch body 106, larger than the diameter of pouch body 106, or smaller than the diameter of pouch body 106, so long as neck portion 118 is able to retain sponge 116 at closed end 110 of pouch 104. Sponge 116 may preferably be between one half inch to over two inches thick in order to obtain the desired length of pouch 104 when sponge collapses. For example, a two-inch-thick sponge can be compressed to a one-half inch thickness by applying pressure on the surface of the sponge by an erect penis. In this case, there is a one and one half inch increase in the length of pouch 104 due to the compression of sponge 116 regardless of any other increase in the length of pouch 104. In addition, the surface of sponge 116 may be provided with a smooth coating to reduce or prevent any abrasion that may result on the penis due to contact with sponge 116. For this purpose, a polyurethane coating may be provided on sponge 116.

In addition, retention sponge 116 may have an insertion opening 120 formed in the outer surface of sponge 116, facing open end 108 of condom 100. Insertion opening 120 is configured to receive a finger of a user to facilitate deployment of condom 100 in a vagina by inserting the sponge with a finger so that no separate insertion tool is required. Furthermore, the provision of insertion opening 120 and the presence of sponge 116 in sponge cavity 114 ensure that a user does not accidentally penetrate closed end 110 of condom 100 during insertion.

Thus, condom 100 may be inserted into vagina 18, and positioned as illustrated in FIG. 2, by using a finger to insert sponge 116. Sponge 116 acts as a retaining means for pouch 104, and causes condom 100 to be retained securely within vagina 18. Open end 108 of condom 100 may then receive a penis (not shown) during sexual intercourse. Open end 108 initially starts as a generally triangular opening, as illustrated in FIG. 3a, due to the shape of frame 102, and transitions into tubular body 106, which is shown in a circular or cylindrical shape in the in the preferred embodiment, but which may be any desired cross-sectional shape. Convolutions 112 are formed along the length of tubular body 106, and serve to retain condom 100 in a contracted configuration, as illustrated in FIG. 3b, while also providing stimulation to the penis during sexual intercourse, and enabling conformance of pouch 104 to the inner wall of vagina 18.

A normal vaginal canal length is two to three inches, but penetration by a penis at the time of intercourse, can increase the length of the vaginal canal to six or seven inches depending on the anatomy of the sexual organs of the partners. The present invention is constructed with a length of two to three inches between outer frame 102 and closed end 110 in the pre-deployment stage. As illustrated in FIG. 3c, when a penis (not shown) is inserted into open end 108 of condom 100, the penis will contact sponge 116, and either compress sponge 116 or push sponge 116 further into the vagina. As sponge 116 is pushed further into the vagina, pouch 104 will stretch in the longitudinal direction as illustrated by arrow 122. Because the invention intends the penis to contact the retaining means, and because the retaining means can move back and forth within the vagina, a sponge 116 or similar soft, non-abrasive, non-chaffing anchoring device is preferred as the retaining means. This is an advantage of the present invention over a number of the prior art devices that rely on rings or similar anchoring mechanisms. Furthermore, as sponge 116 is contacted, convolutions 112 enable pouch 104 to stretch, and as pouch 104 stretches, convolutions 112 will flatten out. Thus, in the same manner as the vaginal canal elongates during intercourse, pouch 104 will also elongate. When the vaginal canal shortens due to withdrawal of the penis, pouch 104 will also shorten in the longitudinal direction opposite to arrow 122.

This shortening of pouch 104 can be due solely to a spring-like action cause by convolutions 112 drawing pouch 104 back to its original deployed configuration, illustrated in FIG. 2. However, as also illustrated in FIGS. 3a-3c, an elastic biasing member or spring 124 may be attached to pouch 104 for facilitating and enhancing return to the original contracted deployed configuration. Thus one or more elastic members 124 may be attached to pouch body 106 for augmenting the resilient return of pouch 104 to its original contracted configuration. As also illustrated in FIG. 4, elastic member 124 may be as simple as a thin 5-10 mm wide, and two to three-inch long strip of latex or other elastic material. First end 126 and second end 128 of elastic member 124 may be attached to tubular body 106 by any suitable means, such as adhesive, heat bonding, molding, or the like. Elastic member 124 may be attached on the inside of tubular body 106 by attaching near to open end 108 and neck 118, and, preferably, two elastic members are attached, one each on opposite sides of the interior of tubular body 106, as illustrated in FIG. 3a. Of course, any number of elastic members can be used with the condom of the invention, so long as the overall effect is sufficient to enable proper elongation of pouch 104 to an elongated configuration during penetration and return of pouch 104 to its contracted deployed configuration upon withdrawal. Other known types of elastic or spring-like members may be substituted for elastic member strip 124 so long as they function to provide a contractive bias in the longitudinal direction to return pouch 104 to a contracted configuration following elongation. Furthermore, the elastic members 124 may be formed as an integral part of pouch 104 while pouch 104 is being manufactured. In other words, the pouch 104 may be formed with a region of elasticity that can be located anywhere as long as it functions to return pouch 104 to a contracted configuration following elongation. Any number of such regions of elasticity may be provided in pouch 104 or even throughout the entirety of pouch 104 for retracting pouch 104 to its original configuration following elongation.

Elastic member 124 can greatly increase the longitudinal retractive bias of condom 100 for returning the condom to its original contracted configuration. Thus, elastic member can retain pouch 104 at a length of about two to three inches, while the actual fully extended length of pouch 104 might be five, six, seven, or greater inches. This built-in longitudinal spring bias in combination with the retention properties of sponge 116 causes outer frame 102 to remain in contact with the female anatomy surrounding the vagina, as illustrated in FIG. 2, rather than slouching out of the vagina, as in the prior art. This secure anchoring of outer frame 102 prevents blocked entry or condom-bypass entry.

In addition, triangularly-shaped outer frame 102 can also have a spring-biased contour associated with it. This enables outer frame 102 to flex so as to better retain contact with the vulva of the female when condom 100 is in vagina 18, and provides additional contractive bias to pouch 104 in the longitudinal direction. Outer frame 102 is preferably sufficiently resilient to flatten out during penetration by the male to provide more intimate contact during intercourse, as illustrated in FIG. 3c, and then return to its original shape following withdrawal. Of course, pouch 104 and elastic member 124 of the invention may also be used with a non-contoured outer frame, such as a flat frame, or a frame shaped in a shape other than triangular, such as the circular frame used in some prior art female condoms.

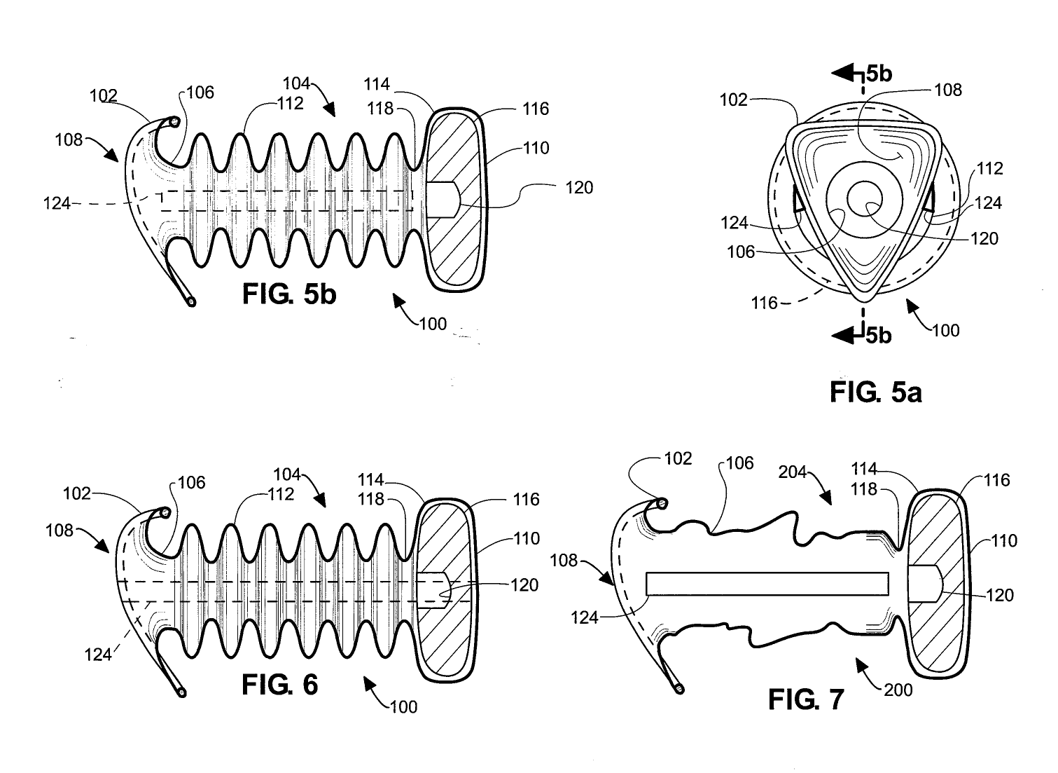

FIGS. 5a and 5b illustrate an alternative embodiment of the invention in which elastic members 124 are attached to the exterior of pouch 104 rather than to the interior. In yet another alternative embodiment, as illustrated in FIG. 6, elastic members 124 may extend the full length of condom 100 from frame 102 to closed end 110. Of course, alternate attachment configurations will be apparent to those skilled in the art, and the disclosed embodiments are only exemplary.

FIG. 7 illustrates an alternative embodiment of a condom 200 in which features identical to those in the above-discussed embodiment are given like item numbers. Condom 200 includes a pouch 204 which, rather than being convoluted, is just bunched up into a contracted configuration. Pouch 204 is retained in this configuration by elastic members 124, which function as described above with respect to embodiment 100 illustrated in FIGS. 3a-3c. As with the prior embodiments, elastic members 124 may be attached to pouch 204, or formed integrally therewith, so long as they function to bias pouch 204 to retract longitudinally toward a contracted configuration. Accordingly, no further discussion of this embodiment is believed necessary.

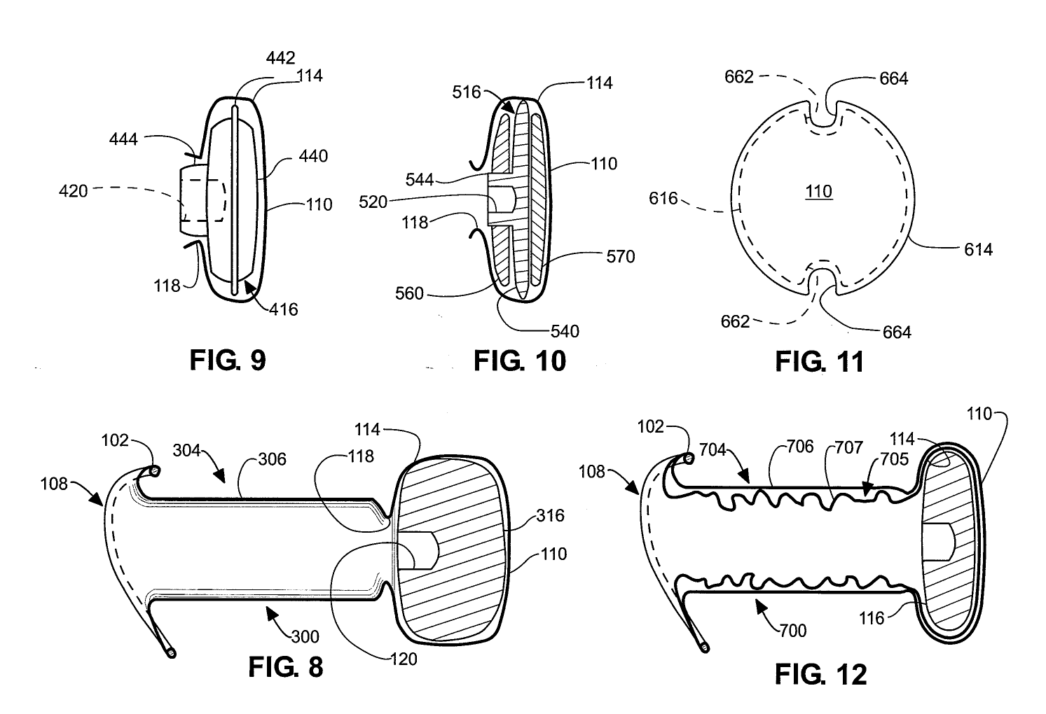

FIG. 8 shows an alternative embodiment of a condom 300 having a pouch 304 with a non-convoluted tubular body 306, and an extra-thick sponge 316. Sponge 316 may be anywhere from one half inch thick to over two inches thick in the longitudinal direction. In this embodiment, compression of sponge 316 serves, at least in part, the same function as elongation of pouch 104 in the embodiment of FIGS. 3a-3c. Thus, as a penis is inserted into condom 300, and the penis contacts sponge 316, sponge 316 will compress substantially and pouch 304 will elongate only slightly, while still functioning in a manner similar to the embodiments discussed above. Alternatively or additionally, in the embodiment of FIG. 8, tubular body 306 of pouch 304 may be formed of highly resilient latex or similar material capable of stretching at least in the longitudinal direction when a penis is inserted, and then springing back to the original contracted configuration when the penis is withdrawn, without requiring attachment of elastic members 124. This highly-elastic tubular body 306 configuration may also be used with a smaller sponge 116 and sponge cavity 114, as illustrated in FIGS. 3a-3c, in place of the extra thick sponge 316.

FIGS. 9-11 illustrate preferred embodiments of sponges for use with the condom of the invention. FIG. 9 illustrates a one-piece sponge 416 having a generally circular body 440, and a thin circular edge 442 that extends about the circumference of circular body 440 for aiding in retaining sponge 416 within a vagina. A circular bulge 444 is located on the side of sponge 416 that will face an open end of a condom of the invention. An insertion opening 420 is formed in bulge 444, and bulge 444 preferably matches the opening of neck 118 so that a user's finger does not slip past sponge 416 and penetrate closed end 110 during insertion, and to further prevent a penis from entering sponge cavity 114 during sexual intercourse.

FIG. 10 illustrates a configuration in which three sponges are placed in sponge cavity 114 in a stacked configuration. A center sponge 516 is formed having a circular sponge body 540 and a thin profile. A circular bulge 544 is of a configuration similar to bulge 444, described above, with an insertion opening 520 formed integrally with sponge 540. An outer sponge 560 and an inner sponge 570 of diameters less than that of sponge 516 are located on either side of sponge 516. Outer sponge 560 includes a hole through it that accommodates bulge 544.

FIG. 11 illustrates a sponge 616 and a sponge cavity 614 viewed from the closed end 110 of a condom of the invention, and having opposing notches 662 formed in sponge 616 and matching opposed notches 664 formed in sponge cavity 614. Notches 662, 664 enable sponge 616 to fold along an axis passing through notches 662, 664, thereby facilitating insertion of sponge 616 into a vagina, and further facilitating retention of sponge 616 in the vagina by inhibiting pullout.

In order for the condom of the invention to function properly, it is very important to ensure that it is pinhole free. In this regard, as illustrated in FIG. 12, the present inventors envision a female condom 700 having a first pouch 704 that contains a second pouch 705 therein. First pouch 704 has a tubular body 706 that is relatively thin and short in length (about 2.5-3.0 inches, for example) having high elasticity at least in the longitudinal direction, while second pouch 705 is relatively thicker and longer (about 5-7 inches, for example), but has substantially less elasticity than first pouch 704. Preferably, second pouch 705 is located inside first pouch 704, and tubular body 707 of second pouch 705 may be bunched up, in a manner similar to pouch 204 of the embodiment illustrated in FIG. 7, may be convoluted, as discussed above with respect to pouch 104 of the embodiment illustrated in FIGS. 3a-3c, or otherwise compacted so as to fit within the substantially shorter first pouch 104.

Both pouches 704, 705 share the same outer frame 108 and define substantially the same sponge cavity 114 for retaining a sponge 116. With this structure, first pouch 704 serves as a longitudinal elastic member to provide resiliency and longitudinal contractive bias to return first pouch 704 and second pouch 705 to a contracted configuration after being elongated. Second pouch 705, being thicker and substantially less elastic than first pouch 704, reduces the chances of failure of the condom due to a pinhole, tear, or the like.

While specific embodiments have been illustrated and described in this specification, those of ordinary skill in the art appreciate that any arrangement that is calculated to achieve the same purpose may be substituted for the specific embodiments disclosed. This disclosure is intended to cover any and all adaptations or variations of the present invention, and it is to be understood that the above description has been made in an illustrative fashion, and not a restrictive one. Combinations of the above embodiments, and other embodiments not specifically described herein will be apparent to those of skill in the art upon reviewing the foregoing disclosure. The scope of the invention should properly be determined with reference to the appended claims, along with the full range of equivalents to which such claims are entitled.

The invention claimed is:

1. A female condom comprising:
a pouch having a closed end and an open end, said pouch being constructed to reside in a contracted configuration when said condom is deployed in a vagina, said pouch further being able to resiliently extend longitudinally from said contracted configuration to an elongated configuration when a penis is inserted into said condom, and said pouch including a longitudinal biasing means for returning said pouch to said contracted configuration following removal of the penis.

2. The condom of claim 1 wherein said contracted configuration is less than three inches and said elongated configuration is more than five inches.

3. The condom of claim 1 further including a frame connected to said open end of said pouch, said frame being generally triangular in shape.

4. The condom of claim 1 further including a retaining means located near said closed end of said pouch for retaining the condom in a vagina.

5. The condom of claim 4 wherein said retaining means is a sponge retained within a sponge cavity.

6. The condom of claim 1 wherein said pouch includes a plurality of convolutions along the length thereof.

7. The condom of claim 1 wherein said pouch includes at least one elastic member attached to said pouch for biasing said pouch into said contracted configuration.

8. The condom of claim 6 wherein said pouch includes at least one elastic member attached to said pouch for biasing said pouch into said contracted configuration.

9. A female condom for deployment in a vagina, said condom comprising:
a frame for contacting the female anatomy external to the vagina;
a pouch connected to said frame, said pouch having an open end and a closed end; and
means for retaining said pouch within the vagina, wherein said pouch includes a biasing means for retaining said pouch in a longitudinally contracted configuration following deployment of said condom in the vagina,
wherein the structure of said biasing means allows said pouch to extend longitudinally, and exerts a contractive longitudinal bias for returning said pouch to said longitudinally contracted configuration.

10. The condom of claim 9 wherein said pouch includes convolutions along the length thereof.

11. The condom of claim 9 wherein said pouch includes at least one elastic member for exerting a longitudinal contractive bias on said pouch.

12. The condom of claim 9 wherein said frame is contoured concavely on the side that contacts the female anatomy surrounding the vagina.

13. The condom of claim 12 wherein said frame is formed of a resilient material that exerts a spring bias on said pouch.

14. The condom of claim 9 wherein said means for retaining said pouch in the vagina is a circular sponge.

15. The condom of claim 9 wherein said pouch is an inner pouch and further including an outer pouch, said inner pouch being located within said outer pouch and said outer pouch being formed of a highly elastic material which exerts said contractive longitudinal bias.

16. A female condom for providing prophylactic protection to a female during sexual intercourse by deployment in the vagina of the female, said condom comprising:
a pouch having an open end, a closed end and a tubular body extending longitudinally between said open end and said closed end, said tubular body having a wall with an interior and an exterior;
a frame for attachment to said open end, said frame supporting said open end to provide an opening into the interior of said tubular body; and
retaining means for retaining said closed end in the vagina, wherein said retaining means is located near said closed end of said pouch and said pouch includes means for elastically biasing said pouch into a contracted configuration.

17. The condom of claim 16 wherein said retaining means is a sponge positioned within said pouch to be directly contacted by the penis of a male during sexual intercourse, whereby said pouch is elongated by such contact.

18. The condom of claim 17 wherein said means for elastically biasing said pouch is at least one elastic member on said pouch which exerts a longitudinal bias for retracting said pouch to a contracted configuration.

19. The condom of claim 18 wherein said elastic member is formed integrally with said pouch.

20. The condom of claim 18 wherein said pouch is a first pouch and said elastic member is a second highly elastic pouch surrounding said first pouch.
